(19) United States
(12) Patent Application Publication
(10) Pub. No.: US 2005/0148944 A1
(43) Pub. Date: Jul. 7, 2005

(54) Syringe safety sleeve

(76) Inventors: Hsin-Po Hsieh, Chian-Hwa Hsien (TW); Chi-Zer Ho, Taipei (TW); Shih-Chun Wang, Chia-Yi (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/743,773

(22) Filed: Dec. 24, 2003

(51) Int. Cl. A61M 5/32

(52) U.S. Cl. 604/198

(57) ABSTRACT

A syringe safety sleeve has an inner sleeve and an outer sleeve. The inner sleeve selectively attaches to a conventional syringe and is slidably mounted inside the outer sleeve. The outer sleeve has an open proximal end and an open distal end. The inner sleeve has an open proximal end, an open distal end and a barrel connector formed on the open distal end. The open proximal end of the outer sleeve is selectively connected to the open proximal end of the inner sleeve when the outer sleeve is pull out from the inner sleeve. The barrel connector of the inner sleeve attaches to a conventional syringe. The syringe safety sleeve can accommodate ordinary syringes with no safety device to protect the used needle. The syringe safety sleeve can be conveniently connected to the syringe and has a simple structure.

### BACKGROUND OF THE INVENTION

1. Field of the Invention

The present invention relates to a safety feature for a syringe, and more particularly to a syringe safety sleeve that can be mounted around the barrel of a syringe and keep people from being injured by the needle of the syringe.

2. Description of Related Art

A conventional syringe comprises a hollow barrel, a plunger and a needle hub with a needle. To keep people from being injured by a used needle, manufacturers have developed safety syringes with retractable needles. In one configuration, the contaminated needle is drawn entirely into the barrel after the syringe has been used. In another configuration, a safety sleeve is formed with the syringe when the syringe is manufactured. The conventional safety sleeve comprises an outer sleeve and an inner sleeve slidably mounted inside the outer sleeve. The inner sleeve of the conventional safety sleeve is formed with the syringe, and the outer sleeve can pull out from the inner sleeve to retainer the used needle.

However, conventional syringes with retractable needles or the safety sleeve formed with the syringe are directly modified during manufacturing and are expensive to manufacture. Furthermore, modified syringes cannot be used with ordinary syringes that have no safety device to protect the used needle.

### SUMMARY OF THE INVENTION

The main objective of the present invention is to provide a syringe safety sleeve that is compatible with ordinary syringes with a simple structure and no safety device to cover used needles of the syringes.

To achieve the objective, a syringe safety sleeve in accordance with the present invention comprises an inner sleeve and an outer sleeve. The inner sleeve slidably mounted inside the outer sleeve and securely attaches to the barrel of a conventional syringe. The syringe safety sleeve can readily convert ordinary syringes with no safety device to protect the used needle to a safety syringe.

Further benefits and advantages of the present invention will become apparent after a careful reading of the detailed description with appropriate reference to the accompanying drawings.

### BRIEF DESCRIPTION OF THE DRAWINGS

FIG. 1 is an exploded perspective view of a syringe safety sleeve in accordance with the present invention and a conventional syringe;

FIG. 2 is a perspective view of the syringe safety sleeve in FIG. 1 mounted on a conventional syringe;

FIG. 3 is a side plan view in partial section of the syringe safety sleeve and conventional syringe in FIG. 2;

FIG. 4 is a perspective view of the syringe safety sleeve and conventional syringe in FIG. 1 with the outer sleeve pulled out of the inner sleeve; and

FIG. 5 is a side plan view in partial section of the syringe safety sleeve and conventional syringe in FIG. 4.

### DETAILED DESCRIPTION OF THE INVENTION

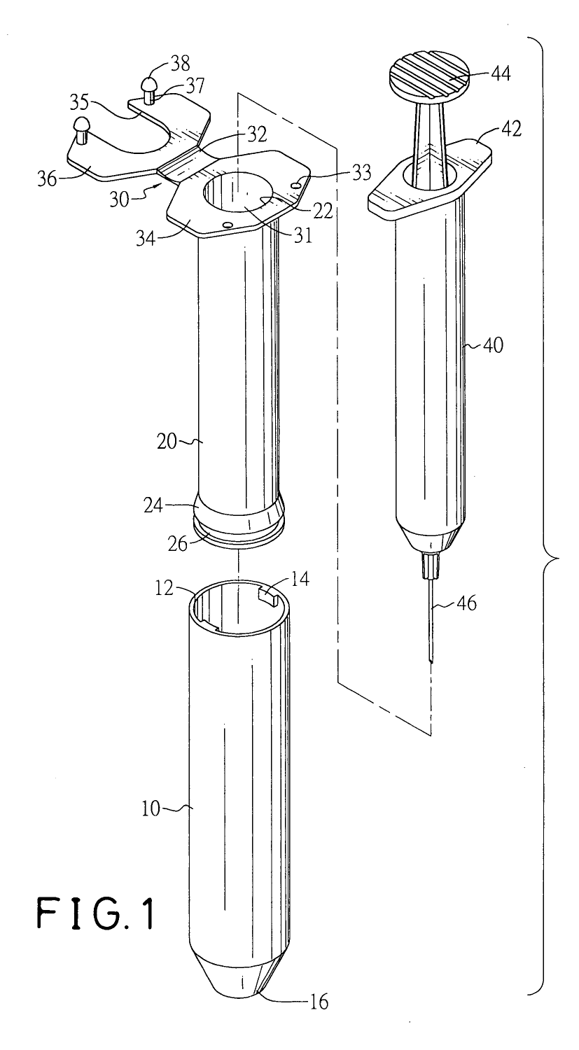

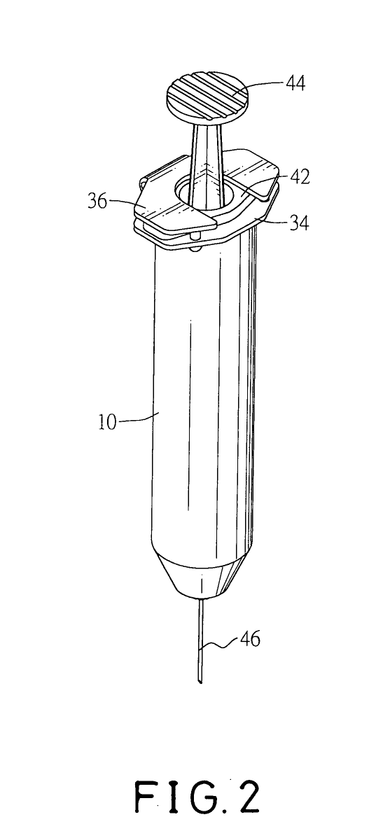

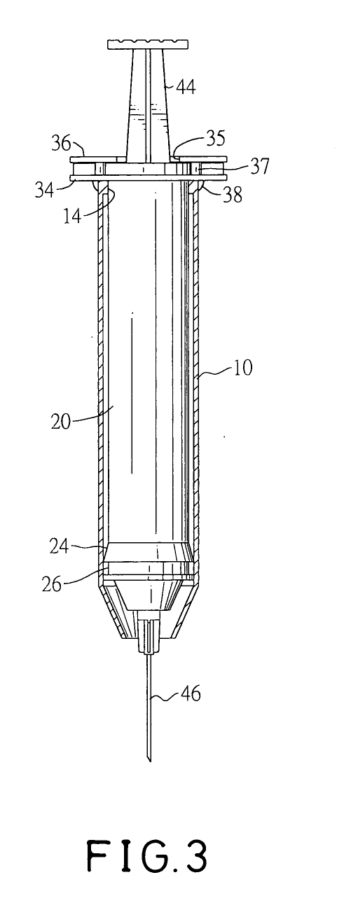

With reference to FIGS. 1 to 3, a syringe safety sleeve in accordance with the present invention is mounted on a conventional syringe (not numbered) to convert the conventional syringe to a safety syringe. The syringe comprises a hollow barrel (40), a plunger (44), a needle hub (not numbered), a needle (46) and a safety cover (not shown). The hollow barrel (40) has a proximal end (not numbered), a distal end (not numbered) and a finger flange (42). The finger flange (42) is formed integrally with and extends radially out from the proximal end of the hollow barrel (40). Before the syringe is used, the safety cover (not shown) covers the needle (46) and attaches to the needle hub.

The syringe safety sleeve comprises an outer sleeve (10) and an inner sleeve (20). The outer sleeve (10) has an open proximal end (12), an open distal end (16) and a locking member (not numbered). The locking member is formed at the open proximal end (12) and may be implemented optionally with multiple protrusions (14) or an annular rib (not shown) extended inward from the open proximal end (12). The open distal end (16) may be tapered inward.

The inner sleeve (20) is slidably mounted inside the outer sleeve (10) and has an open proximal end (24), an open distal end (22), a barrel connector (30) and a locking unit (not numbered). The open proximal end (24) is tapered outward and selectively connects to the open proximal end (12) of the outer sleeve (10) by pulling the outer sleeve (10) out from the inner sleeve (20) until the locking member engages the locking unit. The locking unit is formed at the open proximal end (24) and may be implemented with an annular recess (26) corresponding to the locking member.

The barrel connector (30) is formed on the open distal end (22) of the inner sleeve (20) to connect the inner sleeve (20) to a conventional syringe and has a retainer (36), a hinge (32) and a flange (34). The flange (34) has a center (not numbered), a connected side (not numbered), an unconnected side (not numbered), a bottom (not numbered), a central hole (31) and optional multiple fastener holders (not numbered) and is formed on and protrudes radially from the open distal end (22) of the inner sleeve (20). The central hole (31) is formed in the center of the flange (34). The fastener holders in the flange (34) are formed near the unconnected side of the flange (34) and may be multiple through holes (33), slots (not shown), notches (not shown), etc.

The retainer (36) is selectively connected to the flange (34) and has a connected side (not numbered), an unconnected side (not numbered), a notch (35) and multiple fasteners (not numbered). The notch (35) is shaped to accommodate free movement of the syringe plunger (44) and communicates with the unconnected side of the retainer (36). The fasteners are formed on and protrude from the bottom near the unconnected side, correspond respectively to the fastener holders and attach to the flange (34) to attach the syringe safety sleeve to the finger flange (42) of a conventional syringe. The fasteners are formed near the unconnected side of the retainer (36) corresponding respectively to the fastener holders in the flange (34) and may be multiple cylindrical protrusions (37), hooks (not shown), tabs (not shown), etc. Where the fasteners are implemented with the cylindrical protrusions (37), each cylindrical protrusion (37) has an enlarged head (38). Where the fasteners are implemented with tabs, the tabs may be T-shaped to fit into and be held by slots or notches in the flange (34), or the tabs may be foldable to fold around the unconnected side of the flange (34).

The hinge (32) pivotally connects the connected side of the retainer (36) to the connected side of the flange (34). The hinge (32) has two sides (not numbered). The sides are securely attached respectively to the connected sides of the retainer (36) and the flange (34). The hinge (32) may be a mechanical hinge (not shown) or a flexible tab (not numbered).

Before the syringe safety sleeve is mounted on a syringe, the open proximal end (24) of the inner sleeve (20) is compressed and inserted through the open proximal end (12) into the outer sleeve (10).

To mount the syringe safety sleeve on a syringe, the needle (46) with the safety cover is inserted into the central hole (31) in the flange (34) of the barrel connector (30). The barrel (40) of the syringe is pushed into the inner sleeve (20) until the finger flange (42) abuts the flange (34) on the barrel connector (30), and the barrel connector (30) is fastened around the finger flange (42) of the syringe and securely holds the syringe in the inner sleeve (20). Where the fasteners on the retainer (36) are cylindrical protrusions (37) with enlarged heads (38), the cylindrical protrusions (37) with the enlarged heads (38) are pushed respectively through the corresponding through holes (33) in the flange (34) or are slid through corresponding slots in the flange (34). When the finger flange (42) of the syringe is securely mounted in the connector (30) on the inner sleeve (20), the needle hub of the syringe extends out of the open distal end (16) of the outer sleeve (10) when the open proximal end (12) of the outer sleeve (10) abuts the flange (34) on the inner sleeve (20).

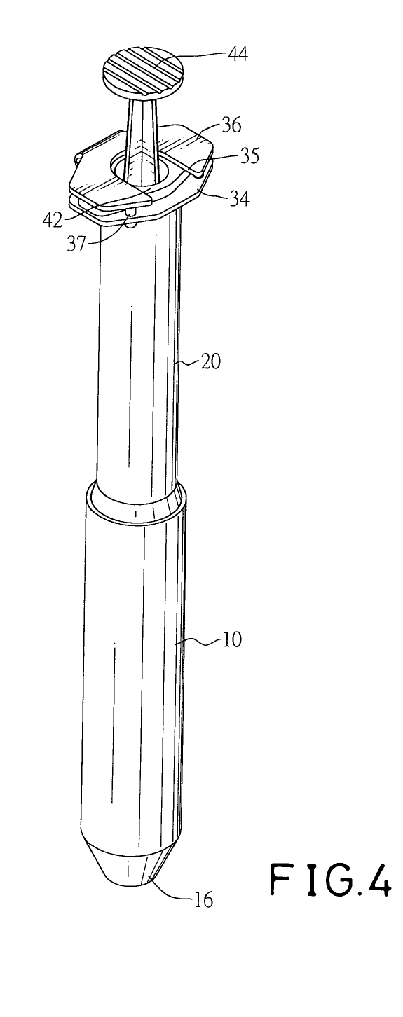

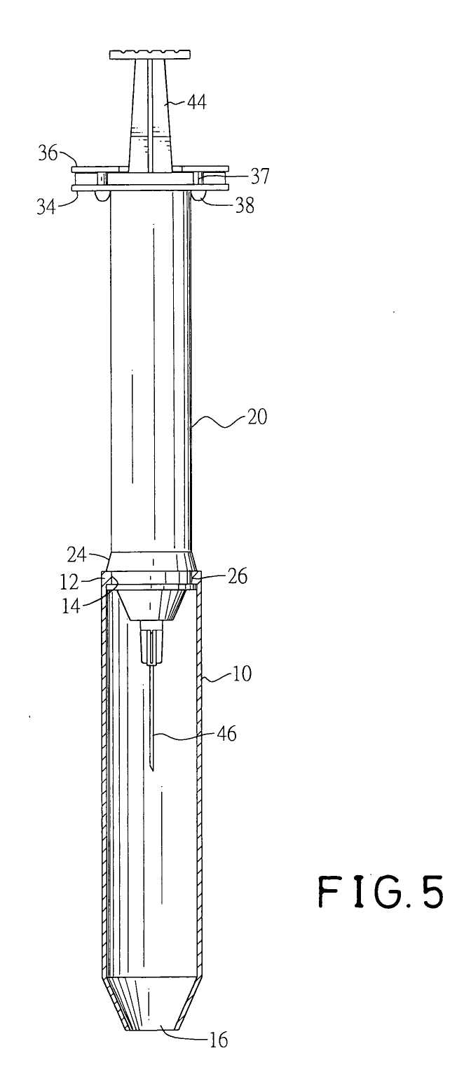

With further reference to FIGS. 4 and 5, to prevent personnel from being injured and infected by contaminated needles (46) during the disposal process, the outer sleeve (10) is pull out from the inner sleeve (20) and locked in position to cover the contaminated needle (46). The open proximal end (12) of the outer sleeve (10) is locked to the open proximal end (24) of the inner sleeve (20) when the locking member on the outer sleeve (10) engages the locking unit on the inner sleeve (20).

The syringe safety sleeve for the syringe as described can accommodate an ordinary syringe with no safety device to protect people from used needle. The syringe safety sleeve can be conveniently connected to the syringe and has a simple structure.

Although the invention has been explained in relation to its preferred embodiment, many other possible modifications and variations can be made without departing from the spirit and scope of the invention as hereinafter claimed.

1. A syringe safety sleeve mounted on a syringe comprising a hollow barrel having a proximal end, a distal end and a finger flange formed integrally with and extending radially out from the proximal end, a plunger, a needle hub, a needle and a safety cover covering the needle and attached to the needle hub before the syringe is used, the syringe safety sleeve comprising:
an outer sleeve having
an open proximal end;
an open distal end; and
a locking member formed on the open proximal end; and
an inner sleeve slidably mounted inside the outer sleeve and having
an open proximal end being tapered outward and selectively connected to the outer sleeve by pulling the outer sleeve out from the inner sleeve;
an open distal end;
a locking unit formed at the open proximal end and corresponding to the locking member; and
a barrel connector formed on the open distal end of the inner sleeve to connect the inner sleeve to a conventional syringe

2. The syringe safety sleeve as claimed in claim 1, wherein the locking member in the outer sleeve is multiple protrusions extending inward from the open proximal end.

3. The syringe safety sleeve as claimed in claim 1, wherein the locking member in the outer sleeve is an annular rib extending inward from the open proximal end.

4. The syringe safety sleeve as claimed in claim 1, wherein the open distal end of the outer sleeve is tapered inward.

5. The syringe safety sleeve as claimed in claim 1, wherein the locking unit is an annular recess corresponding to the locking member.

6. The syringe safety sleeve as claimed in claim 1, wherein the barrel connector has
a flange formed on and protruding radially from the open distal end of the inner sleeve and having
a center;
a connected side;
an unconnected side;
a bottom; and
a central hole formed in the center of the flange;
a retainer selectively connected to the flange and having
a connected side;
an unconnected side; and
a notch shaped to accommodate free movement of the syringe plunger and communicating with the unconnected side; and
multiple fasteners formed near the unconnected side and selectively attached to the flange to connect the syringe safety sleeve to the finger flange of a conventional syringe; and
a hinge that pivotally connects the connected side of the retainer to the connected side of the flange and has two sides securely attached respectively to the connected sides of the retainer and the flange.

7. The syringe safety sleeve as claimed in claim 6, wherein the barrel connector flange has multiple fastener holders formed near the unconnected side.

8. The syringe safety sleeve as claimed in claim 7, wherein the fastener holders in the flange are through holes.

9. The syringe safety sleeve as claimed in claim 7, wherein the fastener holders in the flange are slots.

10. The syringe safety sleeve as claimed in claim 7, wherein the fastener holders in the flange are notches.

11. The syringe safety sleeve as claimed in claim 8, wherein the fasteners on the retainer are cylindrical protrusions with enlarged heads corresponding respectively to the through holes.

12. The syringe safety sleeve as claimed in claim 9, wherein the fasteners on the retainer are cylindrical protrusions with enlarged heads corresponding respectively to the slots.

13. The syringe safety sleeve as claimed in claim 6, wherein the fasteners on the retainer are hooks.

14. The syringe safety sleeve as claimed in claim 6, wherein the fasteners on the retainer are flexible tabs.

15. The syringe safety sleeve as claimed in claim 9, wherein the fasteners on the retainer are T-shaped tabs corresponding respectively to the slots.

16. The syringe safety sleeve as claimed in claim 10, wherein the fasteners on the retainer are T-shaped tabs corresponding respectively to the notches.

17. The syringe safety sleeve as claimed in claim 6, wherein the hinge is a mechanical hinge.

18. The syringe safety sleeve as claimed in claim 6, wherein the hinge is a flexible tab.
